SEQUENCE LISTING (52 sequences)

SEQ ID NO: 1 - DNA, 3754 bp, Artificial Sequence
  full length lacZ cassette sequence
ggtctcgttacagctagctcagtcctaggtattatgctagcagctccatacccgtttttttgggctaacaggaggaattaaccatggggggttctcatcatcatcatcatcatggtatggctagcatgactggtggacagcaaatgggtcgggatctgtacgacgatgacgataaggatccaatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggtaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggcccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaggagacc

SEQ ID NO: 2 - DNA, 909 bp, Artificial Sequence
  PanE crtE
atgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggcccgagggcggttgaagaacgtctgagacaacatcttcagcttgccagtgagcatctctctgcggcctgccaacacgggcacgccactcaacattttattcaggcctggtttgacaaaaaactcgctgccgtcagttaa

SEQ ID NO: 3 - DNA, 912 bp, Artificial Sequence
  PagE crtE
atgatgacggtctgtgcagaacaacacgtcaatttcatacacagcgatgcagccagcctgttgaacgacattgagcaacggcttgatcagcttttaccggttgaaagcgaacgtgacttagtgggcgctgccatgcgcgacggtgcgctggcaccaggaaagcgtatccgtccactgctgttgttgctggcagcgcgcgatctgggctgcaacgccacgcctgccggcctgcttgatctcgcctgcgcggtagagatggtgcatgccgcatcactgattctggatgacatgccctgcatggatgatgcgcaactgcgtcgcggacgtccgaccattcattgccagtatggtgaacatgtcgcgattctggccgcggtggccctgctgagtaaggcattcggcgtggtcgctgcggcagaaggcttaacggcaaccgccagagccgacgctgtagcagaattatcccacgcagtcggcatgcaggggctggtgcaggggcagtttaaggatctctccgaaggtgacaagccacgcagcgctgacgccattctgatgaccaatcactataaaaccagcaccctgttctgcgcctccatgcagatggcttctatcgtggctgaagcctcaggtgaagcccgcgaacagctgcaccgtttttcgcttaatcttggtcaggctttccagctactggacgatctcactgacggcatggccgacaccggtaaagatgcccatcaggatgacgggaaatcaacgctggtgaatctgctggggccacaggcggttgaaacgcgactgcgcgatcatctgcgctgcgccagcgagcatctgttatcggcctgccaggacggttatgccacacaccattttgttcaggcctggtttgagaaaaaactcgctgccgtcagttaa

SEQ ID NO: 4 - DNA, 909 bp, Artificial Sequence
  PvaE crtE
atgacggtctgtgcagaacaacacgtcaatttcatacacagcgatgcagccagcctgttgaacgacattgagcaacggcttgatcagcttttaccggttgaaagcgaacgtgacttagtgggcgctgccatgcgcgacggtgcgctggcaccaggaaagcgtatccgtccactgctgttgttgctggcagcgcgcgatctgggctgcaacgccacgcctgccggcctgctcgatctcgcctgcgcggtagagatggtgcatgccgcatcactgattctggatgacatgccctgcatggatgatgcgcaactgcgtcgcggacgtccgaccattcattgccagtatggtgaacatgtcgcgattctggccgcggtggccctgctgagtaaggcattcggcgtggtcgctgcggcagaaggcttaacggcaaccgccagagccgacgctgtagcagaattatcccacgcagtcggcatgcaggggctggtgcaggggcagtttaaggatctctccgaaggtgacaagccacgcagcgctgacgccattctgatgaccaatcactataaaaccagcaccctgttctgcgcctccatgcagatggcctctatcgtggctgaagcctcaggtgaagcccgcgaacagctgcaccgtttttcgcttaatcttggtcaggctttccagctactggacgatctcactgacggcatggccgacaccggtaaagatgctcatcaggatgacgggaaatcaacgctggtgaatctgctggggccacaggcggttgaaacgcgactgcgcgatcatctgcgctgcgccagcgagcatctgttatcggcctgccgggacggttatgccacacaccattttgttcaggcctggtttgagaaaaaactcgctgccgtcagttaa

SEQ ID NO: 5 - DNA, 879 bp, Artificial Sequence
  RspE crtE
atgaggcacaagatggcgtttgaacagcggattgaagcggcaatggcagcggcgatcgcgcggggccagggctccgaggcgccctcgaagctggcgacggcgctcgactatgcggtgacgcccggcggcgcgcgcatccggcccacgcttctgctcagcgtggccacggcctgcggcgacgaccgcccggctctgtcggacgcggcggcggtggcgcttgagctgatccattgcgcgagcctcgtgcatgacgatctgccctgcttcgacgatgccgagatccggcgcggcaagcccacggtgcatcgcgcctattccgagccgctggcgatcctcaccggcgacagcctgatcgtgatgggcttcgaggtgctggcccgcgccgcggccgaccagccgcagcgggcgctgcagctggtgacggcgctggcggtgcggacggggatgccgatgggcatctgcgcggggcagggctgggagagcgagagccagatcaatctctcggcctatcatcgggccaagaccggcgcgctcttcatcgccgcgacccagatgggcgccattgccgcgggctacgaggccgagccctgggaagagctgggagcccgcatcggcgaggccttccaggtggccgacgacctgcgcgacgcgctctgcgatgccgagacgctgggcaagcccgcggggcaggacgagatccacgcccgcccgaacgcggtgcgcgaatatggcgtcgagggcgcggcgaagcggctgaaggacatcctcggcggcgccatcgcctcgatcccctcctgcccgggcgaggcgatgctggccgagatggtccgccgctatgccgagaagatcgtgccggcgcaggtcgcggcccgcgtctga

SEQ ID NO: 6 - DNA, 930 bp, Artificial Sequence
  PanB crtB
atgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctag

SEQ ID NO: 7 - DNA, 891 bp, Artificial Sequence
  PagB crtB
atggaggtgggatcgaaaagctttgccaccgcgtcaaaactgtttgatgccaaaacccgacgcagcgtgctgatgctctacgcctggtgccgtcactgtgatgatgtgattgacgatcaggtcctgggattcagcaacgatacgccatcgctgcaatctgccgaacagcgcctggcgcagctggagatgaaaacgcgtcaggcctatgccggatcccagatgcatgagcccgcctttgcggcctttcaggaggtggcaatggcgcacgatattctgcctgcttacgcttttgatcatctggcgggctttgcgatggacgtgcatgagacacgctatcagacgctggatgatacgctgcgttactgttaccacgtcgcgggcgtggttggcctgatgatggcgcagattatgggcgtacgcgacaacgccacgctggatcgcgcctgcgatctcggtctggcgtttcagctgaccaatattgcgcgcgatatcgttgaagatgctgaagcgggacgctgctatctgcccgctgcgtggctggctgaagaggggctgacccgagagaatctcgccgatccgcaaaatcgcaaggcattaagccgcgtcgcccgtcggctggtggaaacggcggagccctattatcgatcggcgtcggctggcctgccgggtttaccgctgcgttcagcgtgggcgattgctaccgcgcagcaggtctatcgtaaaatcggtatgaaggtggttcaggcgggttcacaggcgtgggagcaacgccagtccaccagcacgccagagaaactggcactgctggtggcggcatcgggtcaggcggttacttcccgggtggcgcgtcacgctccacgctcagctgatctctggcagcgccccgtttaa

SEQ ID NO: 8 - DNA, 930 bp, Artificial Sequence
  PvaB crtB
atgaatagtccgtcactgctcgatcatgccgtagacaccatggaggtgggatcgaaaagctttgccaccgcgtcaaaactgtttgatgccaaaacccgacgcagcgtgctgatgctctacgcctggtgccgtcactgtgatgatgtgattgacgatcaggtactgggattcagcaacgatacgccatcgctgcaatctgccgaacagcgcctggcgcagctggagatgaaaacacgtcaggcctatgccggatcgcagatgcatgagcccgcctttgcggcctttcaggaggtggcaatggcacacgatattctgcctgcttacgcttttgatcatctggcgggctttgcgatggacgtgcatgagacacgctatcagacgctggatgatacgctgcgttactgttaccacgtcgcgggcgtggttggcctgatgatggcgcagattatgggcgtacgcgacaacgccacgctggatcgcgcctgcgatctcggtctggcgtttcagctgaccaatattgcgcgcgatatcgttgaagatgctgaagcgggacgctgctatctgcccgctgcgtggctggctgaagaggggctgacccgagagaatctcgccgatccgcaaaatcgccaggcactcagccgcgtcgcccgtcggctggtggaaacggcggagccctattatcgatcggcgtcggctggcctgccgggtttaccgctgcgttcagcgtgggcgattgctaccgcgcagcaggtctatcgtaaaatcggtatgaaggtggttcaggcgggttcacaggcgtgggagcaacgccagtccaccagcacgccagagaaactggcactgctggtggcggcatcgggtcaggcggttacttcccgggtggcgcgtcacgctccacgctccgctgatctctggcagcgccccgtttaa

SEQ ID NO: 9 - DNA, 1068 bp, Artificial Sequence
  RspB crtB
atgattgcctctgccgatctcgatgcctgccgggagatgatccgcaccggctcctattccttccatgccgcgtcccgcctgctgcccgagcgcgtgcgcgcgccgtcgctggcgctctatgccttctgccgcgtggccgacgatgcggtcgacgaggcggtgaacgatggacagcgcgaggaggatgccgaggtcaagcgccgcgccgtcctgagcctgcgcgaccggctggacctcgtctatggcggccgcccgcgcaatgcgccggccgaccgcgccttcgccgcggtggtcgaggagttcgagatgccccgggcgctgcccgaggcgctgctcgaggggctcgcctgggacgcggtggggcggagctacgacagtttctcgggcgtgctcgactattcggcgcgggtggccgcggcggtgggggcgatgatgtgcgtcctcatgcgggtgcgcgatcccgacgtgctggcccgggcctgcgatctgggcctcgccatgcagctcaccaacatcgcccgcgacgtggggaccgacgcgcgctcgggacggatctatctgccgcgcgactggatggaggaggaggggctgccggtcgaggagttcctcgcccggccggtggtcgacgaccgcatccgcgcggtgacgcaccgcctgctgcgcgcggccgaccggctctatctgcgttcggaagcgggggtctgcggcctgcctctggcctgccggcccggcatctatgccgcgcgccacatctatgcgggtatcggcgacgagatcgcgcggaacggctatgacagcgtgacgcgccgcgccttcaccacgcggcgccagaagctcgtctggctcgggctctcggccacacgcgcggccctcagcccgttcggccccggctgcgccacgctgcatgcggcgcccgagcccgaagtggccttcctcgtcaatgccgccgcccgggcccggccgcagcgcggccgctccgaggcgctgatctcggttctggcccagctcgaggcgcaggatcggcagatctcgcggcagcgactggggaaccgggccaacccgatctag

SEQ ID NO: 10 - DNA, 1479 bp, Artificial Sequence
  PanI crtI
atgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatatga

SEQ ID NO: 11 - DNA, 1479 bp, Artificial Sequence
  PagI crtI
atgaatagaactacagtaattggcgcaggctttggtggtctggctctggccattcgccttcaggcgtcaggcgttcccacccgactgctggagcagcgtgacaagccgggcggccgggcttatgtctatcaggatcagggcttcacgtttgatgccggccccacggtaatcaccgatcccagcgccattgaagagctgttcactctggcgggtaaaaagctctctgactatgtcgagctgatgccggtgaagccgttttatcgcctctgctgggagtccggcaaggtgttcagttatgacaacgatcagcccgcgctggaagcgcagattgccgcatttaatccgcgtgacgttgaaggatatcggcgctttctggcctattcccgagcggtgtttgctgaaggctatctgaagcttggcaccgtgccgtttctgtcattccgcgacatgctgcgggccgcgcctcagctggcaaaacttcaggcatggcgcagcgtttacagcaaagtggcgagctacattgaagatgagcatctgcgtcaggccttctctttccactcactgctggtgggcggaaatccgtttgccacttcctcaatctataccctgattcatgcgctggaacgtgaatggggcgtctggttcccgcgcggtggcacgggcgcgctggtgcagggcatggtgaaactgtttgaagatctgggcggcgaagtggagctcaatgccagcgttgcccggctggagacccaggaaaacaggattaccgcggtgcacctgaaagatggccgggtcttcccgacccgcgcggttgcctccaacgcagatgtggttcacacctaccgcgaactgctgagccagcaccccgcttcgcaggcgcagggacggtcactgcagaacaaacgcatgagtaactcgctgtttgtgatctattttggcctgaatcatcatcacgatcagctggcgcaccacacggtctgctttggtccgcgctatcgtgagttgattgatgaaatctttaacaaagatggcctggcagaggacttctcgctctatctgcatgcgccctgcgtgaccgatccctcactggcaccggaaggctgcggcagctactacgtgctggcgccggtaccgcacctcggcaccgctgatatcgactgggccgttgaaggtccgcgcctgcgcgatcgcattttcgactatctggaacagcattacatgccgggcctgcgtagccagttggtcacgcatcgcatcttcacgccgtttgatttccgcgatgagctgaatgcgtatcagggctcggccttctcagtggagccgatcctgacgcaaagcgcctggttccggcctcacaaccgcgataaaaatattaataatctctatctggtcggtgctggtacccatcctggcgcgggtattccaggggtgattggctcggccaaggctaccgcaggattgatgctggaggatctggcttga

SEQ ID NO: 12 - DNA, 1487 bp, Artificial Sequence
  PvaI crtI
atgaaacgaactacagtaattggcgcaggctttggtggtctggccctggcaattcgccttcaggcgtcaggcgttcccacccgactgctggagcagcgtgacaagcctggcggccgggcttatgtctatcaggatcagggcttcacgtttgatgccggccccacggtaatcaccgatcccagcgccattgaagagctgttcaccctggcgggtaaaaagctctctgactatgtcgagctgatgccggtgaagccgttttatcgcctctgctgggagtccggcaaggtgttcagttatgacaacgatcagcccgcgctggaagcgcagattgccgcgtttaatccgcgtgacgttgaaggatatcgtcgctttctggcctattcccgagcggtctttgctgaaggctatctgaagcttggcaccgtgccgtttctgtcattccgcgacatgctgcgggccgcgcctcagctggcaaaacttcaggcgtggcgcagcgtttacagcaaagtggcgagctacattgaagatgagcatctgcgtcaggccttctctttccactcactgctggtgggcggaaatccgtttgccacttcctcaatctataccctgattcatgcgctggaacgtgaatggggcgtctggttcccgcgcggtggcacgggcgcgctggtgcagggcatggtgaaactgtttgaagatctgggcggcgaagtggagctcaatgccagcgttgcccggctggaaacccaggaaaacaggattaccgcggtacacctgaaagatggccgggtcttcccaacccgcgcggttgcctccaacgcagatgtggttcacacctaccgcgaactgctgagccagcatcccgcttcgcaggcgcagggacggtcactgcagaacaaacgcatgagcaactcgctgtttgtgatctattttggcctgaatcatcatcacgatcagctggcgcaccacacggtctgctttggtccgcgctatcgtgagttgattgatgagatctttaacaaagatggcctggcagaggacttctcgctctatctgcatgcgccctgcgtgaccgatccctcactggcgccggagggctgcggcagctactacgtgctggcgccagtaccgcacctcggcaccgccgatatcgactgggccgttgaaggtccgcgcctgcgcgatcgcatttttgactatctggaacagcactatatgccgggcctgcgtagccagttggtcacgcatcgcatcttcacgccgtttgatttccgcgatgagctgaatgcgtatcagggttcggccttctcggtggagccgatcctgacgcaaagcgcctggttccggcctcacaaccgcgataaaaatattgataatctctatctggtcggtgcaggtacccatcctggcgcgggtattccaggcgtgattggctcggccaaggctaccgcaggattgatgctggaggatctggcttgattaattaa

SEQ ID NO: 13 - DNA, 1557 bp, Artificial Sequence
  RspI crtI
atgccctcgatctcgcccgcctccgacgccgaccgcgcccttgtgatcggctccggactggggggccttgcggctgcgatgcgcctcggcgccaagggctggcgcgtgacggtcatcgacaagctcgacgttccgggcggccgcggctcctcgatcacgcaggaggggcaccggttcgatctgggacccaccatcgtgacggtgccgcagagcctgcgcgacctgtggaagacctgcgggcgggacttcgacgccgatgtcgagctgaagccgatcgatccgttctacgaggtgcgctggccggacgggtcgcacttcacggtgcgccagtcgaccgaggcgatgaaggccgaggtcgcgcgcctctcgcccggcgatgtggcgggatacgagaagttcctgaaggacagcgaaaagcgctactggttcggttacgaggatctcggccgccgctcgatgcacaagctgtgggatctcatcaaggtgctgcccaccttcgggatgatgcgggccgaccggtcggtctaccagcacgccgcgcttcgggtgaaggacgagcggctgcgcatggcgctctcgttccacccgctcttcatcggcggcgaccccttcaacgtgacctcgatgtatatccttgtgagccagctcgagaaggagttcggcgtccattatgccatcggcggcgtggcggccatcgccgcggccatggcgaaggtgatcgaggggcagggcggcagcttccgcatgaacaccgaggtggacgagatcctcgtcgagaagggcaccgccaccggtgtgcggctcgcctcgggcgaggtgctgcgggcgggtctcgtggtctcgaatgcggatgcgggccatacctacatgcggcttctgcgtaaccatccgcgccgccgctggaccgacgcccatgtgaagagccggcgctggtcgatggggctgttcgtctggtatttcggaacgaaggggacgaagggcatgtggcccgacgtcggccaccacacgatcgtcaatgcgccgcgctacaaggggctggtcgaggacatcttcctcaagggcaagctcgcgaaggacatgagcctctatatccaccggccctcgatcaccgatccgaccgtggcgcccgagggggatgacacgttctatgcgctctcgcccgtgccgcatctgaaacaggcgcaaccggtggactggcaggctgtggccgagccctaccgcgaaagcgtgctcgaggtgctcgaacagtcgatgccggggatcggggaacggatcgggccctcgctcgtcttcacccccgagaccttccgcgaccgctacctcagcccctggggcgcgggcttctcgatcgagccgcggatcctgcagtcggcctggttccggccgcacaacatttccgaggaggtggcgaacctgttcctcgtgggcgcgggcacccatccgggtgcgggcgtgcccggcgtgatcggttcggccgaagtgatggccaagcttgcccccgatgcgccacgtgcgcgccgcgaggccgaacctgctgaaaggcttgccgcggaatga

SEQ ID NO: 14 - DNA, 549 bp, Artificial Sequence
  coding sequence of idi
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 15 - DNA, 4980 bp, Artificial Sequence
  pYC1k-ccdB-idi vector
gacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaattacgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacgtattgagaccggcttactaaaagccagataacagtatgcgtatttgcgcgctgatttttgcggtataagaatatatactgatatgtatacccgaagtatgtcaaaaagaggtatgctatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataataaactagagccaggcatcaaataaaacgaaaggctcagtcgaaagactgggcctttcgttttatctgttgtttgtcggtgaacgctctctactagagtcacactggctcaccttcgggtgggcctttctgcgtttataggtctcgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataactgcagctggtgccgcgcggcagccaccaccaccaccaccactaatacagattaaatcagaacgcagaagcggtctgataaaacagaattaatggcgatgacgcatcctcacgataatatccgggtaggcgcaatcactttcgtctctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatcgtcgacgtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaactgccctgcaaggcggttttttcgttttcagagcaagagattacgcgcagaccaaaacgatctcaagaagatcatcttattaatcagataaaatatttctagatttcagtgcaatttatctcttcaaatgtagcacgcggccgccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgaccgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggg

SEQ ID NO: 16 - DNA, 22 bp, Artificial Sequence
  oligo1-1F
ctataagcatcagacagcactg

SEQ ID NO: 17 - DNA, 22 bp, Artificial Sequence
  oligo1-1R
gtaacagtgctgtctgatgctt

SEQ ID NO: 18 - DNA, 22 bp, Artificial Sequence
  oligo1-2F
ttgaagcttatcggatcgagcc

SEQ ID NO: 19 - DNA, 22 bp, Artificial Sequence
  oligo1-2R
cgccggctcgatccgataagct

SEQ ID NO: 20 - DNA, 22 bp, Artificial Sequence
  oligo1-1F
ctataagcatcagacagcactg

SEQ ID NO: 21 - DNA, 22 bp, Artificial Sequence
  oligo1-1R
gtaacagtgctgtctgatgctt

SEQ ID NO: 22 - DNA, 22 bp, Artificial Sequence
  oligo3-1F
ctgaacggcaagccgttgctga

SEQ ID NO: 23 - DNA, 22 bp, Artificial Sequence
  oligo3-1R
cgaatcagcaacggcttgccgt

SEQ ID NO: 24 - DNA, 22 bp, Artificial Sequence
  oligo3-2F
ggatttttgcatcgagctgggt

SEQ ID NO: 25 - DNA, 22 bp, Artificial Sequence
  oligo3-2R
tattacccagctcgatgcaaaa

SEQ ID NO: 26 - DNA, 22 bp, Artificial Sequence
  oligo1-2F
ttgaagcttatcggatcgagcc

SEQ ID NO: 27 - DNA, 22 bp, Artificial Sequence
  oligo1-2R
cgccggctcgatccgataagct

SEQ ID NO: 28 - DNA, 22 bp, Artificial Sequence
  oligo1-1F
ctataagcatcagacagcactg

SEQ ID NO: 29 - DNA, 22 bp, Artificial Sequence
  oligo1-1R
gtaacagtgctgtctgatgctt

SEQ ID NO: 30 - DNA, 22 bp, Artificial Sequence
  oligo4-1F
tgactacctacgggtaacagtt

SEQ ID NO: 31 - DNA, 22 bp, Artificial Sequence
  oligo4-1R
aagaaactgttacccgtaggta

SEQ ID NO: 32 - DNA, 22 bp, Artificial Sequence
  oligo4-2F
gtttacagggcggcttcgtctg

SEQ ID NO: 33 - DNA, 22 bp, Artificial Sequence
  oligo4-1R
aagaaactgttacccgtaggta

SEQ ID NO: 34 - DNA, 22 bp, Artificial Sequence
  oligo4-2F
gtttacagggcggcttcgtctg

SEQ ID NO: 35 - DNA, 22 bp, Artificial Sequence
  oligo4-2R
gtcccagacgaagccgccctgt

SEQ ID NO: 36 - DNA, 22 bp, Artificial Sequence
  oligo4-3F
gattggcctgaactgccagctg

SEQ ID NO: 37 - DNA, 22 bp, Artificial Sequence
  oligo4-3R
gcgccagctggcagttcaggcc

SEQ ID NO: 38 - DNA, 22 bp, Artificial Sequence
  oligo1-2F
ttgaagcttatcggatcgagcc

SEQ ID NO: 39 - DNA, 22 bp, Artificial Sequence
  oligo1-2R
cgccggctcgatccgataagct

SEQ ID NO: 40 - DNA, 22 bp, Artificial Sequence
  oligo1-1F
ctataagcatcagacagcactg

SEQ ID NO: 41 - DNA, 22 bp, Artificial Sequence
  oligo1-1R
gtaacagtgctgtctgatgctt

SEQ ID NO: 42 - DNA, 22 bp, Artificial Sequence
  oligo5-1F
ttggagtgacggcagttatctg

SEQ ID NO: 43 - DNA, 22 bp, Artificial Sequence
  oligo5-1R
cttccagataactgccgtcact

SEQ ID NO: 44 - DNA, 22 bp, Artificial Sequence
  oligo5-2F
gagcgaacgcgtaacgcgaatg

SEQ ID NO: 45 - DNA, 22 bp, Artificial Sequence
  oligo5-2R
gcaccattcgcgttacgcgttc

SEQ ID NO: 46 - DNA, 22 bp, Artificial Sequence
  oligo5-3F
ctgaactaccgcagccggagag

SEQ ID NO: 47 - DNA, 22 bp, Artificial Sequence
  oligo5-3R
ggcgctctccggctgcggtagt

SEQ ID NO: 48 - DNA, 22 bp, Artificial Sequence
  oligo5-4F
cgcgcgaattgaattatggccc

SEQ ID NO: 49 - DNA, 22 bp, Artificial Sequence
  oligo5-4R
gtgtgggccataattcaattcg

SEQ ID NO: 50 - DNA, 22 bp, Artificial Sequence
  oligo1-2F
ttgaagcttatcggatcgagcc

SEQ ID NO: 51 - DNA, 22 bp, Artificial Sequence
  oligo1-2R
cgccggctcgatccgataagct

SEQ ID NO: 52 - DNA, 2710 bp, Artificial Sequence
  pUC57
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2016/0340670 A1
(43) Pub. Date: Nov. 24, 2016

(54) NOVEL OLIGO-LINKER-MEDIATED DNA ASSEMBLY METHOD AND APPLICATIONS THEREOF

(71) Applicant: Nanjing Jinsirui Science & Technology Biology Corporation, Nanjing City (CN)

(72) Inventors: Chunbo LOU, Nanjing City (CN); Yong TAO, Nanjing City (CN); Xuejin ZHAO, Nanjing City (CN); Shasha ZHANG, Nanjing City (CN); Lihua ZHANG, Nanjing City (CN); Chunhua ZHAI, Nanjing City (CN); Zhenyu LIU, Nanjing City (CN)

(21) Appl. No.: 15/160,426

(22) Filed: May 20, 2016

(30) Foreign Application Priority Data
May 22, 2015 (CN) 201510268154.3

(51) Int. Cl. C12N 15/10; C12N 15/52; C12N 15/70

(52) U.S. Cl. CPC C12N 15/1079; C12N 15/70; C12N 15/52; C12N 2800/101; C12N 2800/50

(57) ABSTRACT

A method for generating a library of expression vectors comprising a plurality of donor sequences and a plurality of oligo-linker nucleic acids, termed Oligonucleotide Linker-Mediated DNA Assembly (OLMA), is described. Also described are applications of the OLMA method, including the simultaneous tuning of several factors in metabolic and biological pathways, and the combinatorial high throughput optimization of metabolic and biological pathways.

### CROSS REFERENCE TO RELATED APPLICATION

This application claims the benefit under 35 U.S.C. §119(a) of Chinese Patent Application Number CN201510268154.3, filed May 22, 2015, the entire disclosure of which is hereby incorporated herein by reference.

### REFERENCE TO SEQUENCE LISTING SUBMITTED ELECTRONICALLY

This application contains a sequence listing, which is submitted electronically via EFS-Web as an ASCII formatted sequence listing with a file name “688096-90US Sequence Listing”, creation date of May 20, 2016, and having a size of 42.2 kb. The sequence listing submitted via EFS-Web is part of the specification and is herein incorporated by reference in its entirety.

### FIELD OF THE INVENTION

The invention is generally in the field of synthetic biology and relates to a method for generating a library of expression vectors comprising a plurality of donor sequences and a plurality of oligo-linker nucleic acids, termed Oligonucleotide Linker-Mediated DNA Assembly (OLMA). Applications of the method, especially applications involving high-throughput and combinatorial optimization of metabolic or biological pathways, are also provided.

### BACKGROUND OF THE INVENTION

Microbes can be used for the production of renewable chemicals in the field of industrial microbiology (Keasling (2010), Science, 330: 1355-8). With the fields of synthetic biology and metabolic engineering rapidly growing, the ability to use microbes as platforms for the production of valuable chemicals has greatly improved (Alper et al. (2005), Nat Biotechnol., 23: 612-6; Juminaga et al. (2012), Appl Environ Microbiol., 78: 89-98; Na et al. (2013), Nat Biotechnol., 31, 170-4; Smanski et al. (2014), Nat Biotechnol., 32: 1241-9).

One bottleneck of these applications is that an imbalanced expression of metabolic enzymes can result in the accumulation of toxic metabolites and therefore inhibit cell growth, resulting in decreased production of the product (Coussement et al. (2014), Metabolic Engineering, 23: 70-7). Therefore, balancing the enzymatic activity and expression level of the relevant enzymes is key for the optimization of metabolic pathways (Farasat et al. (2014), Mol Syst Biol., 10: 731; Jones et al. (2014), Curr Opin Biotechnol., 33: 52-59).

Optimization of the expression level of pathway enzymes can be achieved by the following methods: (1) adjusting gene copy number by changing the plasmid copy number (Jensen and Hammer (1998), Appl Environ Microbiol., 64: 82-7); (2) adjusting gene expression level by introducing regulatory sequences (Salis et al. (2009), Nat Biotechnol., 27: 946-50; Salis (2011), Methods Enzymol., 498: 19-42); (3) changing the order of the genes in the operon (Lim et al. (2011), Proc Nail Acad Sci USA, 108: 10626-31; Nishizaki et al. (2007), Appl Environ Microbiol., 73: 1355-61); and (4) using enzymes from different species with varied enzymatic characteristics and substrate specificities (Rodriguez et al. (2014), Microb Cell Fact., 13: 126).

The DNA sequences involved in expression of metabolic pathway enzymes can be grouped into two categories: long sequences, which are usually more than 200 base pairs (bp) long and contain coding sequences of genes and plasmid replication origins, and short sequences, which are usually less than 50 bp long and contain or encode regulatory sequences such as promoters and ribosome binding site (RBS) sequences. Due to the difficulty of assembling multiple genes, current methods for optimizing gene expression level are mainly limited to the modulation of a single factor at a time. Reports demonstrating the modulation of several factors simultaneously are rare.

Several techniques that have been described recently, including Gibson Assembly and Golden Gate cloning methods, can be used to assemble several DNA pieces in a single reaction (Gibson et al. (2009), Nat Methods, 6: 343-5; Weber et al. (2011), PLoS One, 6: e19722). However, most of these methods are dependent on polymerase chain reactions (PCRs), which can potentially introduce undesired mutations, particularly when amplifying sequences longer than 2 kb. The Golden Gate cloning method does not require the use of PCR to amplify the pieces of DNA, but it introduces barcode sequences to dictate the predefined assembly order. When using Golden Gate cloning to assemble DNA pieces in different orders, each assembled piece must be sub-cloned to introduce different barcoding sequences, resulting in significantly increased reagent and labor costs.

Despite the progress described in the art, there is a need in the art for improved methods for DNA assembly, including a PCR- and barcode-free method for the high-throughput assembly and optimization of DNA libraries, such as a DNA library encoding the enzymatic components of metabolic and biological pathways. Such a method could greatly increase the efficiency of metabolic and biological engineering.

### BRIEF SUMMARY OF THE INVENTION

The invention satisfies this need by providing a PCR- and barcode-free method for DNA library assembly, termed Oligonucleotide Linker-Mediated DNA Assembly (OLMA). The invention also provides a method for high-throughput and combinatorial optimization of the enzymatic components of biological pathways, such as a metabolic pathway, using this OLMA method.

In a general aspect, the invention relates to a method for generating a library of expression vectors comprising a plurality of donor sequences. The method comprises:

(a) obtaining a plurality of donor vectors, each independently comprising: (i) a first cleavage site recognizable by a type IIS restriction endonuclease, (ii) a donor sequence, and (iii) a second cleavage site recognizable by the type IIS restriction endonuclease, wherein upon digestion with the type IIS restriction endonuclease, the plurality of donor vectors will provide a plurality of double-stranded donor nucleic acid fragments, each independently comprising: (i) a donor 5′ overhang, (ii) a donor sequence, and (iii) a donor 3′ overhang, and the donor 5′ overhang and the donor 3′ overhang are not complementary to each other;

(b) providing an entry vector comprising a selectable marker gene and a first cleavage site and a second cleavage site recognizable by the type IIS restriction endonuclease, wherein upon digestion with the type IIS restriction endonuclease, the entry vector will provide an entry vector backbone comprising: (i) an entry vector 5′ overhang, (ii) an entry vector backbone comprising the selectable marker gene, and (iii) an entry vector 3′ overhang;

(c) providing a plurality of chemically synthesized double-stranded oligo-linker nucleic acid molecules, each independently comprising: (i) a linker 5′ overhang, (ii) a linker sequence, and (iii) a linker 3′ overhang, wherein the linker 5′ overhang is complementary to at least one of the donor 3′ overhangs or to the entry vector 3′ overhang, and the linker 3′ overhang is complementary to at least one of the donor 5′ overhangs or to the entry vector 5′ overhang;

(d) mixing (i) the plurality of donor vectors, (ii) the plurality of double-stranded oligo-linker nucleic acid molecules, (iii) the entry vector, (iv) the type IIS restriction endonuclease, and (v) a ligase, in a reaction mixture; and

(e) incubating the reaction mixture under a condition to assemble the library of expression vectors.

According to particular embodiments, the method further comprises:

(f) treating the library of expression vectors with DNase; and

(g) transforming the DNase-treated library of expression vectors into competent cells.

According to particular embodiments, the plurality of donor vectors comprise at least 2 donor sequences, and the plurality of double-stranded oligo-linker nucleic acid molecules comprises at least 2 linker sequences.

According to particular embodiments, the plurality of donor vectors and the entry vector do not contain additional cleavage sites recognizable by the type IIS restriction endonuclease. For example, additional cleavage sites recognizable by the type IIS restriction endonuclease located within the donor vectors and the entry vector are removed by mutagenesis.

According to particular embodiments, each of the donor 5′ overhang, the linker 5′ overhang, the entry vector 5′ overhang, the donor 3′ overhang, the linker 3′ overhang and the entry vector 3′ overhang has 4 nucleotides.

According to particular embodiments, each of the donor DNA sequences comprises at least 200 base pairs. In particular embodiments, each of the donor DNA sequences comprises coding sequences of genes or plasmid origin of replication sequences.

According to particular embodiments, each of the double-stranded oligo-linker nucleic acid molecules comprises no more than 50 base pairs. In particular embodiments, each of the double-stranded oligo-linker nucleic acid molecules comprises a pair of phosphorylated chemically synthesized oligonucleotides. In other particular embodiments, each of the double-stranded oligo-linker nucleic acid molecules comprises regulatory sequences, such as promoter or ribosome binding site sequences.

According to particular embodiments, the assembly reaction condition in step (e) comprises: (i) 10 cycles of 5 minutes at 37° C. followed by 10 minutes at 16° C.; (ii) 15 minutes at 37° C.; (iii) 5 minutes at 50° C.; and (iv) 5 minutes at 80° C.

In another general aspect, the invention relates to a system for generating a library of expression vectors comprising a plurality of donor sequences, the system comprising:

(a) a plurality of donor vectors, each independently comprising: (i) a first cleavage site recognizable by a type IIS restriction endonuclease, (ii) a donor sequence, and (iii) a second cleavage site recognizable by the type IIS restriction endonuclease, wherein upon digestion with the type IIS restriction endonuclease, the plurality of donor vectors will provide a plurality of double-stranded donor nucleic acid fragments, each independently comprising: (i) a donor 5′ overhang, (ii) a donor sequence, and (iii) a donor 3′ overhang, and the donor 5′ overhang and the donor 3′ overhang are not complementary to each other;

(b) an entry vector comprising a selectable marker gene and a first cleavage site and a second cleavage site recognizable by the type IIS restriction endonuclease, wherein upon digestion with the type IIS restriction endonuclease, the entry vector will provide an entry vector backbone comprising: (i) an entry vector 5′ overhang, (ii) an entry vector backbone comprising the selectable marker gene, and (iii) an entry vector 3′ overhang;

(c) a plurality of chemically synthesized double-stranded oligo-linker nucleic acid molecules, each independently comprising: (i) a linker 5′ overhang, (ii) a linker sequence, and (iii) a linker 3′ overhang, wherein the linker 5′ overhang is complementary to at least one of the donor 3′ overhangs or to the entry vector 3′ overhang, and the linker 3′ overhang is complementary to at least one of the donor 5′ overhangs or to the entry vector 5′ overhang; and

(d) the type IIS restriction endonuclease and a ligase to be mixed and incubated with the plurality of donor vectors, the plurality of double-stranded oligo-linker nucleic acid molecules, and the entry vector for the assembly of the library of expression vectors.

According to particular embodiments, the system further comprises DNase.

In another general aspect, the invention relates to a method of optimizing a biological pathway, comprising:

(a) generating a library of expression vectors using a method of the invention, wherein the library comprises a plurality of genes of the biological pathway or variants thereof as the donor sequences, and a plurality of regulatory sequences as the linker sequences;

(b) transforming the library of expression vectors into a host cell; and

(c) identifying clones having the optimized biological pathway from the transformed cells.

According to particular embodiments, the biological pathway is a metabolic pathway.

According to particular embodiments, the library of expression vectors comprises the genes or variants thereof and the regulatory sequences in various assembly orders. According to other particular embodiments of the invention, the library of expression vectors comprises various variants of the genes and/or various variants of the regulatory sequences.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

In the drawings:

FIG. 1 shows the preparation steps for an method according to an embodiment of the invention, e.g., an OLMA method of DNA library assembly;

FIG. 2 shows the assembly step of the OLMA method of DNA library assembly;

FIG. 3 shows how the lacZ cassette (a) was divided into three (b), four (c) or five (d) pieces to test the OLMA method of DNA library assembly;

FIG. 4 shows the donor vectors with their overhang sequences used for the assembly of crtE, crtB and crtI genes from different species;

FIG. 5 shows the oligo-linker nucleic acid molecules designed to serve as linkers for the assembly of the components of the lycopene metabolic pathway in different gene orders; and

FIG. 6 shows the vector map of the pYC1k-ccdB-idi entry vector.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

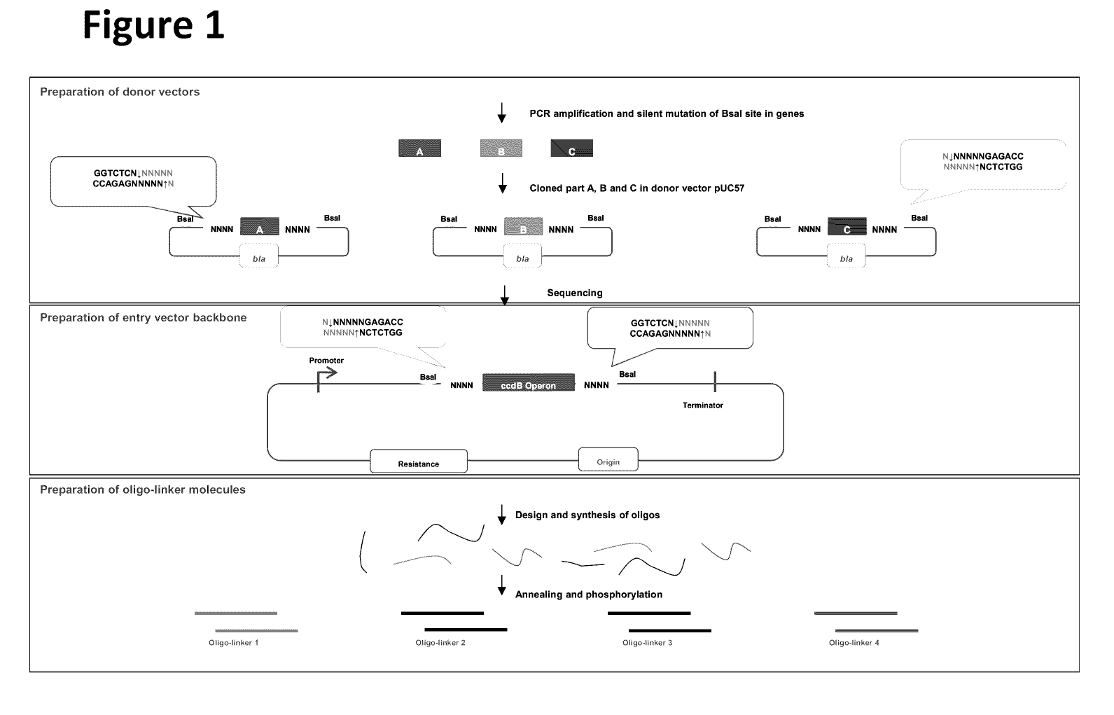

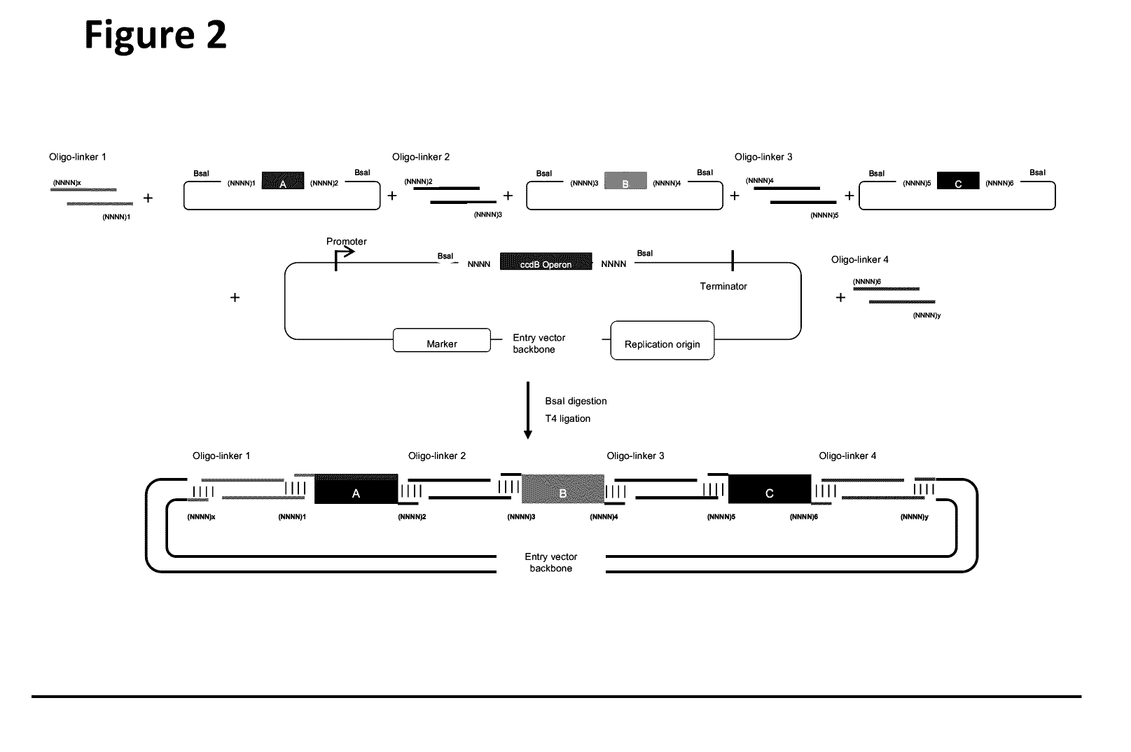

The invention relates to a novel method for generating a library of expression vectors, termed Oligonucleotide Linker-Mediated DNA Assembly (OLMA), wherein a combinatorial library can be generated in a PCR- and barcode-free manner. The preparation steps for an OLMA method for DNA library assembly according to an embodiment of the invention are illustrated in FIG. 1, and the assembly step of the OLMA method for DNA library assembly is illustrated in FIG. 2.

In a general aspect, the invention relates to a method for generating a library of expression vectors comprising a plurality of donor sequences. The method comprises:

(a) obtaining a plurality of donor vectors, each independently comprising: (i) a first cleavage site recognizable by a type IIS restriction endonuclease, (ii) a donor sequence, and (iii) a second cleavage site recognizable by the type IIS restriction endonuclease, wherein upon digestion with the type IIS restriction endonuclease, the plurality of donor vectors will provide a plurality of double-stranded donor nucleic acid fragments, each independently comprising: (i) a donor 5′ overhang, (ii) a donor sequence, and (iii) a donor 3′ overhang, and the donor 5′ overhang and the donor 3′ overhang are not complementary to each other;

(b) providing an entry vector comprising a selectable marker gene and a first cleavage site and a second cleavage site recognizable by the type IIS restriction endonuclease, wherein upon digestion with the type IIS restriction endonuclease, the entry vector will provide an entry vector backbone comprising: (i) an entry vector 5′ overhang, (ii) an entry vector backbone comprising the selectable marker gene, and (iii) an entry vector 3′ overhang;

(c) providing a plurality of chemically synthesized double-stranded oligo-linker nucleic acid molecules, each independently comprising: (i) a linker 5′ overhang, (ii) a linker sequence, and (iii) a linker 3′ overhang, wherein the linker 5′ overhang is complementary to at least one of the donor 3′ overhangs or to the entry vector 3′ overhang, and the linker 3′ overhang is complementary to at least one of the donor 5′ overhangs or to the entry vector 5′ overhang;

(d) mixing (i) the plurality of donor vectors, (ii) the plurality of double-stranded oligo-linker nucleic acid molecules, (iii) the entry vector, (iv) the type IIS restriction endonuclease, and (v) a ligase, in a reaction mixture; and

(e) incubating the reaction mixture under a condition to assemble the library of expression vectors.

As used herein, the term “plurality” means more than one. In particular embodiments, the plurality of donor vectors or the plurality of chemically-synthesized double-stranded oligo-linker nucleic acid molecules comprise at least two donor vectors and at least two chemically-synthesized double-stranded oligo-linker nucleic acid molecules. In more particular embodiments, the plurality of donor vectors or the plurality of chemically-synthesized double-stranded oligo-linker nucleic acid molecules comprise two, three, four, five, six, seven, eight, nine, ten or more donor vectors and two, three, four, five, six, seven, eight, nine, ten or more chemically-synthesized double-stranded oligo-linker nucleic acid molecules.

As used herein, the term “donor sequence” refers to a DNA sequence that is at least 200 bp long. A donor sequence can be any DNA sequence that is 200 bp or longer. In particular embodiments, a donor sequence comprises a coding sequence for a polypeptide, a regulatory noncoding sequence, or fragments thereof. In other particular embodiments, a donor sequence comprises a plasmid origin of replication. The donor sequence can be a gene sequence, a fragment thereof, or a variant hereof.

According to particular embodiments, a plurality of donor sequences comprise variants of a gene coding sequence, including, but not limited to, homologs from different species, mutants, fragments, or other variants. The variants can encode polypeptide that have, for example, different solubility, stability, kinetic properties, substrate specificity, etc. than the parent polypeptide. In particular embodiments, all variants of a particular donor sequence comprise the same set of 5′ and 3′ overhangs.

As used herein, the terms “donor vector backbone” and “donor vector” are used interchangeably and refer to the vector backbone comprising: (i) a first cleavage site recognizable by a type IIS restriction endonuclease, (ii) a donor sequence, and (iii) a second cleavage site recognizable by the type IIS restriction endonuclease. In particular embodiments, the plurality of donor vectors provide a plurality of double-stranded donor nucleic acid fragments upon digestion with the type IIS restriction endonuclease, and each of the double-stranded donor nucleic acid fragments comprises independently: (i) a donor 5′ overhang, (ii) a donor sequence, and (iii) a donor 3′ overhang, and the donor 5′ overhang and the donor 3′ overhang are not complementary to each other. In particular embodiments, the overhangs are 4 bp long.

The donor vector backbones can comprise any vector backbones suitable for molecular cloning manipulation. In particular embodiments, the donor vector backbones comprise the pUC57 vector, the pUC18 vector, or pET series vectors.

As used herein, the term “type IIS restriction endonuclease” refers to restriction endonucleases that cleave DNA at a defined distance from their non-palindromic asymmetric recognition sites. A type IIS restriction endonuclease can be any type IIS restriction endonuclease. In particular embodiments, the type IIS restriction endonuclease cleaves DNA 4 base pairs away from its recognition site. In particular embodiments, the type IIS restriction endonuclease comprises BbvI, BcoDI, BsmAI, BsmFI, FokI, SfaNI, BbsI, BfuAI, BsaI, BsmBI, BspMI, BtgZI, BaeI, SgeI, BslFI, BsoMAI, Bst71I, FaqI, AceIII, BbvII, BveI, or BplI. In more particular embodiments, the type IIS restriction endonuclease is BsaI.

According to particular embodiments, the plurality of donor vectors and the entry vector do not contain additional cleavage sites recognizable by the type IIS restriction endonuclease. For example, additional cleavage sites recognizable by the type IIS restriction endonuclease located within the donor vectors and the entry vector are removed by mutagenesis. In particular embodiments, silent mutations are introduced into the sites of additional cleavage sites recognizable by the type IIS restriction endonuclease to remove the sites. The mutagenesis is carried out using known methods in the art, such as PCR mutagenesis or gene synthesis, in view of the present disclosure.

As used herein, the term “silent mutation” refers to a change of a nucleotide within a gene sequence that does not result in a change in the coded amino acid sequence.

As used herein, the terms “oligo-linker nucleic acid molecule” and “oligo-linker molecule” are used interchangeably and refer to a DNA sequence that is 50 base pairs or fewer long. Accordingly, the oligo-linker nucleic acid molecule can be any DNA sequence that is 50 base pairs or fewer long. In particular embodiments, the oligo-linker nucleic acid molecule comprises (i) a linker 5′ overhang, (ii) a linker sequence, and (iii) a linker 3′ overhang. In particular embodiments, the overhangs are 4 bp long.

In particular embodiments, an oligo-linker nucleic acid molecule comprises or encodes a regulatory sequence, including but not limited to, a promoter, an operator, a ribosome binding site (RBS), a combination of a promoter and an RBS, a terminator, an insulator, or a variant thereof.

According to particular embodiments, the plurality of double-stranded oligo-linker nucleic acid molecules comprise or encode variations of regulatory elements, including, but not limited to, promoters, operators, or RBS, with varying strengths.

The double-stranded oligo-linker nucleic acid molecules can be obtained using methods in the art in view of the present disclosure. In particular embodiments, the double-stranded oligo-linker nucleic acid molecules are generated by annealing a pair of complementary forward and reverse single-stranded oligonucleotides. In other particular embodiments, the resulting double-stranded oligo-linker nucleic acid molecules are phosphorylated using known methods in the art. In particular embodiments, the double-stranded oligo-linker nucleic acid molecules are phosphorylated using T4 polynucleotide kinase (NEB, Cat. No. M0201L).

In particular embodiments, the complementary forward and reverse oligonucleotides comprise chemically synthesized primers that are generated using known methods in the art.

As used herein, the term “linker sequence” refers to an oligo-linker nucleic acid molecule that connects two sequences. In particular embodiments, an oligo-linker nucleic acid molecule connects two donor sequences, e.g., through its 5′ and 3′ overhangs, which are complementary to the 3′ overhang of the upstream donor sequence and to the 5′ overhang of the downstream donor sequence, respectively. In other particular embodiments, an oligo-linker nucleic acid molecule connects a donor sequence to the entry vector backbone, e.g., through the oligo-linker nucleic acid molecule's 5′ and 3′ overhangs, which are complementary to the 3′ overhang of the upstream donor sequence and the 5′ overhang of the entry vector backbone, respectively, or the 3′ overhang of the entry vector backbone and the 5′ overhang of the downstream donor sequence, respectively.

As used herein, the term “complementary” refers to the hybridization or base-pairing between nucleotides or nucleic acids, such as, for instance, that which occurs between the two strands of a double stranded DNA molecule.

According to particular embodiments, the order of the donor sequences is varied by varying the sequence of the 5′ and 3′ overhangs on the double-stranded oligo-linker nucleic acid molecules.

According to particular embodiments, at least two of the donor sequences, the oligo-linker molecules, and the assembly order of the donor sequences are varied simultaneously to produce high throughput combinatorial libraries. According to other particularly embodiments, the donor sequences, the oligo-linker molecules, and the assembly order of the donor sequences are varied simultaneously to produce high throughput combinatorial libraries.

As used herein, the terms “entry vector backbone” and “entry vector” are used interchangeably and refer to the vector backbone into which the assembled nucleic acid, generated by an OLMA method of the invention, is cloned. In particular embodiments, the entry vector comprises a selectable marker gene and a first and second cleavage site recognizable by the type IIS restriction endonuclease such that, upon digestion with the type IIS restriction endonuclease, the entry vector backbone will provide an entry vector backbone comprising: (i) an entry vector 5′ overhang, (ii) an entry vector backbone comprising a selectable marker gene, and (iii) an entry vector 3′ overhang. In particular embodiments, the overhangs are 4 bp long.

The entry vector backbone can comprise any vector backbones suitable for molecular cloning manipulation. In particular embodiments, the entry vectors comprise the pYC1k vector or other vectors with the replication origin of pSC101 or p15A replication origin.

As used herein, the term “selectable marker gene” refers to a gene that is detectable upon its expression in a cell, due to a specific property of the encoded protein. In particular embodiments, the selectable marker gene confers resistance to an antibiotic or drug to the cell in which the selectable marker is expressed. In more particular embodiments, selectable marker genes include, but are not limited to the kanamycin resistance gene, the ampicillin resistance gene, the tetracycline resistance gene, the chloramphenicol resistance gene, and the streptomycin resistance gene.

As used herein, the terms “ligase” and “DNA ligase” are used interchangeably and refer to a family of enzymes which catalyze the formation of a covalent phosphodiester bond between two distinct DNA strands, i.e. a ligation reaction. Accordingly, the ligase that is used to assemble the long and short double-stranded nucleic acid fragments can be any DNA ligase. In particular embodiments, the DNA ligase is T4 DNA ligase.

According to embodiments of the invention, a library of expression vectors comprising a plurality of donor sequences is generated by preparing a reaction mixture comprising: (1) a plurality of donor vectors, (ii) a plurality of double-stranded oligo-linker nucleic acid molecules, (iii) an entry vector, (iv) a type IIS restriction endonuclease, and (v) a ligase, in a reaction mixture, and incubating the reaction mixture under a condition to assemble the library of expression vectors. The reaction mixture can be incubated under any condition suitable for the reactions of the type IIS restriction endonuclease and the ligase. In particular embodiments, the reaction mixture is incubated under a condition comprising: (i) 10 cycles of 5 minutes at 37° C. followed by 10 minutes at 16° C., (ii) 15 minutes at 37° C., (iii) 5 minutes at 50° C., and (iv) 5 minutes at 80° C.

According to particular embodiments, the method further comprises, after the assembly step, the following steps:

(f) treating the library of expression vectors with DNase; and

(g) transforming the DNase-treated library of expression vectors into competent cells.

The DNase can be any DNase. In particular embodiments, the DNase is from a commercially available kit, and the protocol provided in the manual is followed. In more particular embodiments, the DNase is Plasmid-Safe™ ATP-dependent DNase (Epicentre, Cat. No. 3101K).

The competent cells can be any high efficiency competent cells, such as DH5α competent cells.

In another general aspect, the invention relates to a system for generating a library of expression vectors comprising a plurality of donor sequences, the system comprising:

(a) a plurality of donor vectors, each independently comprising: (i) a first cleavage site recognizable by a type IIS restriction endonuclease, (ii) a donor sequence, and (iii) a second cleavage site recognizable by the type IIS restriction endonuclease, wherein upon digestion with the type 11S restriction endonuclease, the plurality of donor vectors will provide a plurality of double-stranded donor nucleic acid fragments, each independently comprising: (i) a donor 5′ overhang, (ii) a donor sequence, and (iii) a donor 3′ overhang, and the donor 5′ overhang and the donor 3′ overhang are not complementary to each other;

(b) an entry vector comprising a selectable marker gene and a first cleavage site and a second cleavage site recognizable by the type IIS restriction endonuclease, wherein upon digestion with the type IIS restriction endonuclease, the entry vector will provide an entry vector backbone comprising: (i) an entry vector 5′ overhang, (ii) an entry vector backbone comprising the selectable marker gene, and (iii) an entry vector 3′ overhang;

(c) a plurality of chemically synthesized double-stranded oligo-linker nucleic acid molecules, each independently comprising: (i) a linker 5′ overhang, (ii) a linker sequence, and (iii) a linker 3′ overhang, wherein the linker 5′ overhang is complementary to at least one of the donor 3′ overhangs or to the entry vector 3′ overhang, and the linker 3′ overhang is complementary to at least one of the donor 5′ overhangs or to the entry vector 5′ overhang; and

(d) the type IIS restriction endonuclease and a ligase to be mixed and incubated with the plurality of donor vectors, the plurality of double-stranded oligo-linker nucleic acid molecules, and the entry vector for the assembly of the library of expression vectors.

According to particular embodiments, the system further comprises DNase.

In another general aspect, the invention relates to a method optimization of a biological pathway, comprising:

a) generating a library of expression vectors using a method of the invention, wherein the library comprises a plurality of genes of the biological pathway or variants thereof as the donor sequences, and a plurality of regulatory sequences as the linker sequences;

(b) transforming the library of expression vectors into a host cell; and

(c) identifying clones having the optimized biological pathway from the transformed cells.

Any biological pathway can be optimized by a method of the invention. The clones containing optimized biological pathway of interest can be selected and/or screened using methods known in the art in view of the present disclosure. In particular embodiments, the biological pathway is a metabolic pathway, more particularly, a metabolic pathway for the lycopene production. Different clones displayed levels of lycopene production can be identified, e.g., by different intensities of red coloring on an indicator plate. The method of optimization can be conducted in a high-through put fashion using methods known in the art in view of the present disclosure.

The host cell used for bacterial expression can be any strains used for bacterial expression, such as DH5α, BL21(DE3), JM109, or MG1655.

Different from the prior art methods for the assembly of a library of expression vectors or optimization of a biological pathway, an OLMA method provided in this invention has at least the following unique advantages and features:

(1) the OLMA method uses double-stranded oligo-linker nucleic acid molecules to facilitate the assembly of donor sequences, by both linking and dictating the assembly order of the donor sequences, and to introduce regulatory sequences to tunc gene expression level;

(2) the OLMA method uses type IIS restriction endonucleases, which cut outside of their recognition site, for seamless assembly—the 4 bp overhangs on the donor sequences that are released by restriction digestion of the donor vectors and the overhangs on the oligo-linker nucleic acid molecules determine the assembly order, which can be easily changed by changing the overhangs on the oligo-linker nucleic acid molecules;

(3) the gene expression level can be modulated using the OLMA method by simultaneously tuning multiple factors in a pathway, such as a metabolic pathway, including (a) using enzyme coding genes from different species, or variants thereof, (b) introducing regulatory sequences, such as RBS sequences with varied strengths, and (c) changing the assembly order of the genes. Combinatorial libraries can be generated by varying any or all of these factors in about 10 days. The resulting combinatorial libraries can be screened to assess gene expression level optimization;

(4) PCR amplification is not required by the OLMA method, which makes it possible to avoid the introduction of mutations generated by PCR-amplification of long DNA sequences; and

(5) the OLMA method involves a one-tube and one-step assembly step, which can save labor and reagent costs.

### EXAMPLES

The following examples of the invention are to further illustrate the nature of the invention. It should be understood that the following examples do not limit the invention and that the scope of the invention is to be determined by the appended claims.

The experimental methods used in the following examples, unless otherwise indicated, are all ordinary methods. The reagents used in the following embodiments, unless otherwise indicated, are all purchased from ordinary reagent suppliers.

### Example 1

### Assembly of the lacZ Gene from E. coli Strain EG1655 Using the OLMA Method of DNA Assembly

The lacZ gene from E. coli was assembled using the OLMA method to assess the efficiency of the method for assembling donor sequences and double-stranded oligo-linker nucleic acid molecules. In this example, the donor sequences comprised pieces of the lacZ coding sequence, and the double-stranded oligo-linker nucleic acid molecules, which were less than 50 bp long, comprised pieces of the lacZ coding sequence.

The E. coli DH5α strain (TransGen Biotech) was used for molecular cloning manipulation, and the E. coil DB3.1 strain, which carries the gyrA462 mutation, was used for the propagation of plasmids containing the ccdB operon. All strains were grown at 37° C. LB medium with 50 μg/ml kanamycin was used to propagate plasmids containing the ccdB operon and the pUC57 plasmid.

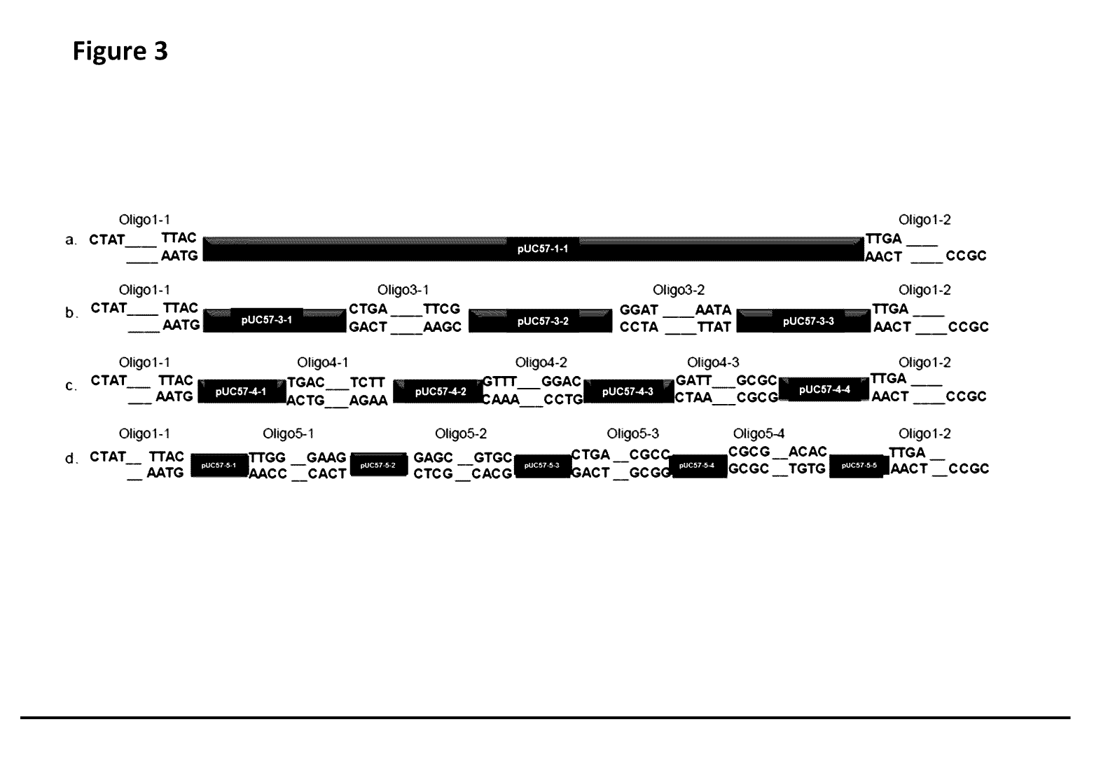

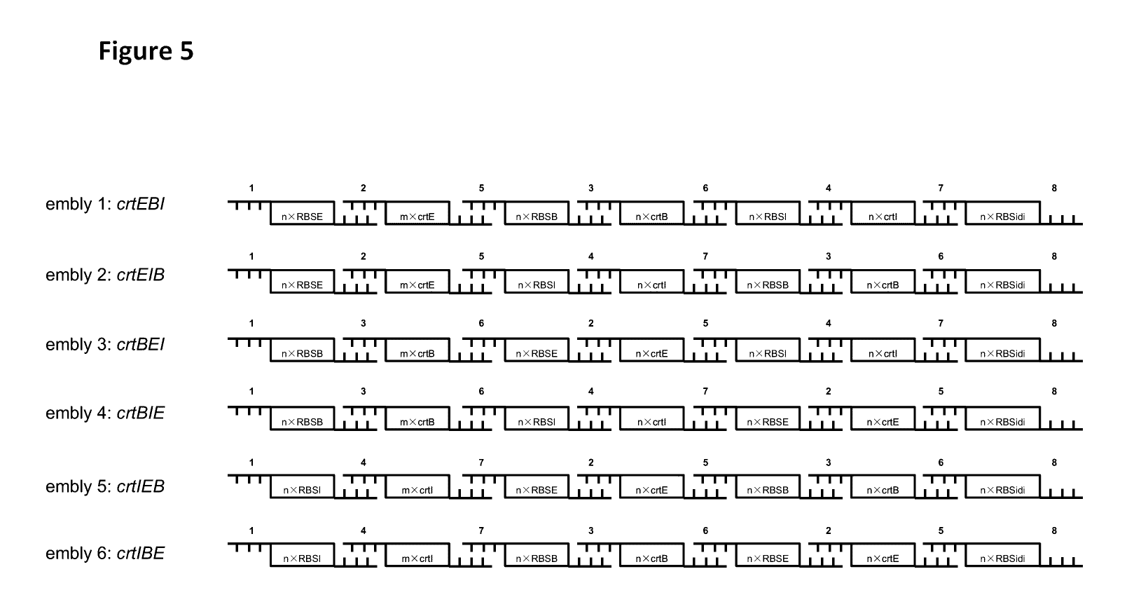

The lacZ gene coding sequence was from the genome of E. coli strain EG1655. The full length lacZ cassette sequence is illustrated in SEQ ID NO: 1 (3.7 kb), and it comprises the constitutive promoter pJ23101, the LacZ coding sequence (Genbank No. 945006), and the rrnB terminator. The full length cassette was cloned into the pUC57 vector (SEQ ID NO: 52). The lacZ cassette was flanked by two BsaI recognition sites, which generated different overhangs for subsequent assembly (FIG. 3a). The full length lacZ cassette was divided into 7, 9, or 11 pieces, consisting of 3 donor sequences plus 4 double-stranded oligo-linker nucleic acid molecules (FIG. 3b), 4 donor sequences plus 5 double-stranded oligo-linker nucleic acid molecules (FIGS. 3c), and 5 donor sequences plus 6 double-stranded oligo-linker nucleic acid molecules (FIG. 3d), respectively. The donor sequences were flanked by BsaI cutting sites on either side and were cloned into donor vectors. The donor sequences from the donor vectors were assembled, along with the double-stranded oligo-linker nucleic acid molecules, into full length lacZ cassettes.

Short oligos were designed to serve as double-stranded oligo-linker nucleic acid molecules based on the OLMA method. For each assembly, (n+1) pairs of short oligos were required to assemble n different donor sequences. Adjacent sequences (donor sequences comprising gene pieces and double-stranded oligo-linker nucleic acid molecules) shared complementary overhangs, ensuring that the sequences would be assembled in a predefined order. The oligo sequences used for the assembly of the lacZ cassette are shown in Table 1. Full-length assembly of the lacZ cassette, resulting in lacZ expression, gives rise to the formation of blue colonies on plates containing IPTG and X-gal, allowing the cassette assembly efficiency to be determined. The results indicate that the efficiency for assembling 3, 7, 9, and 11 pieces was 99.9%, 95%, 43%, and 10%, respectively.

TABLE 1
Short oligos designed to serve as
double-stranded oligo-linker nucleic
acid molecules for the assembly of
the lacZ cassette using the OLMA method

| name | sequence | purpose |
|---|---|---|
| oligo1-1F | SEQ ID NO: 16 | Used for the |
| | CTATAAGCATCAGACAGCACTG | assembly of 3 |
| oligo1-1R | SEQ ID NO: 17 | pieces, as |
| | GTAACAGTGCTGTCTGATGCTT | depicted in |
| Oligo1-2F | SEQ ID NO: 18 | FIG. 3a |
| | TTGAAGCTTATCGGATCGAGCC | |
| Oligo1-2R | SEQ ID NO: 19 | |
| | CGCCGGCTCGATCCGATAAGCT | |
| oligo1-1F | SEQ ID NO: 20 | Used for the |
| | CTATAAGCATCAGACAGCACTG | assembly of 7 |
| oligo1-1R | SEQ ID NO: 21 | pieces, as |
| | GTAACAGTGCTGTCTGATGCTT | depicted in |
| Oligo3-1F | SEQ ID NO: 22 | FIG. 3b |
| | CTGAACGGCAAGCCGTTGCTGA | |
| Oligo3-1R | SEQ ID NO: 23 | |
| | CGAATCAGCAACGGCTTGCCGT | |
| Oligo3-2F | SEQ ID NO: 24 | |
| | GGATTITTGCATCGAGCTGGGT | |
| Oligo3-2R | SEQ ID NO: 25 | |
| | TATTACCCAGCTCGATGCAAAA | |
| Oligo1-2F | SEQ ID NO: 26 | |
| | ITGAAGCTTATCGGATCGAGCC | |
| Oligo1-2R | SEQ ID NO: 27 | |
| | CGCCGGCTCGATCCGATAAGCT | |
| oligo1-1F | SEQ ID NO: 28 | Used for the |
| | CTATAAGCATCAGACAGCACTG | assembly of 9 |
| oligo1-1R | SEQ ID NO: 29 | pieces, as |
| | GTAACAGTGCTGTCTGATGCTT | depicted in |
| Oligo4-1F | SEQ ID NO: 30 | FIG. 3c |
| | TGACTACCTACGGGTAACAGTT | |
| Oligo4-1R | SEQ ID NO: 31 | |
| | AAGAAACTGTTACCCGTAGGTA | |
| Oligo4-2F | SEQ ID NO: 32 | |
| | GTTTACAGGGCGGCTTCGTCTG | |
| Oligo4-1R | SEQ ID NO: 33 | |
| | AAGAAACTGTTACCCGTAGGTA | |
| Oligo4-2F | SEQ ID NO: 34 | |
| | GTTTACAGGGCGGCTTCGTCTG | |
| Oligo4-2R | SEQ ID NO: 35 | |
| | GTCCCAGACGAAGCCGCCCTGT | |
| Oligo4-3F | SEQ ID NO: 36 | |
| | GATTGGCCTGAACTGCCAGCTG | |
| Oligo4-3R | SEQ ID NO: 37 | |
| | GCGCCAGCTGGCAGTTCAGGCC | |
| Oligo1-2F | SEQ ID NO: 38 | |
| | TTGAAGCTTATCGGATCGAGCC | |
| Oligo1-2R | SEQ ID NO: 39 | |
| | CGCCGGCTCGATCCGATAAGCT | |
| oligo1-1F | SEQ ID NO: 40 | Used for the |
| | CTATAAGCATCAGACAGCACTG | assembly of 11 |
| oligo1-1R | SEQ ID NO: 41 | pieces, as |
| | GTAACAGTGCTGTCTGATGCTT | depicted in |
| Oligo5-1F | SEQ ID NO: 42 | FIG. 3d |
| | TTGGAGTGACGGCAGTTATCTG | |
| Oligo5-1R | SEQ ID NO: 43 | |
| | CTTCCAGATAACTGCCGTCACT | |
| Oligo5-2F | SEQ ID NO: 44 | |
| | GAGCGAACGCGIAACGCGAATG | |
| Oligo5-2R | SEQ ID NO: 45 | |
| | GCACCATTCGCGTTACGCGTTC | |
| Oligo5-3F | SEQ ID NO: 46 | |
| | CTGAACTACCGCAGCCGGAGAG | |
| Oligo5-3R | SEQ ID NO: 47 | |
| | GGCGCTCTCCGGCTGCGGTAGT | |
| Oligo5-4F | SEQ ID NO: 48 | |
| | CGCGCGAATTGAATTATGGCCC | |
| Oligo5-4R | SEQ ID NO: 49 | |
| | GTGTGGGCCATAATTCAATTCG | |
| Oligo1-2F | SEQ ID NO: 50 | |
| | TTGAAGCTTATCGGATCGAGCC | |
| Oligo1-2R | SEQ ID NO: 51 | |
| | CGCCGGCTCGATCCGATAAGCT | |

### Example 2

### Optimization of Lycopene Biosynthetic Pathways by Sonstructing a Combinatorial Library Using an OLMA Method of DNA Library Assembly

In this example, the donor sequences comprised coding sequences from different genes, and the double-stranded oligo-linker nucleic acid molecules encoded RBS sequences.

The E. coli DH5α strain (TransGen Biotech) was used for molecular cloning manipulation, and the E. coli DB3.1 strain, which carries the gyrA462 mutation, was used for the propagation of plasmids containing the ccdB operon. All strains were grown at 37° C. LB medium with 50 μg/ml kanamycin was used to propagate plasmids containing the ccdB operon and the pUC57 plasmid.

The E. coil DH5α strain (TransGen Biotech) was used for molecular cloning manipulation, and the E. coli Trans-TI strain (TransGen Biotech) was used were purchased from TransGen Biotech.

The lycopene biosynthetic pathway comprises four key genes: crtE, crtB, crtI, and idi. Versions of each of crtE, crtB and crtI were chosen from the four following species: Pantoea ananatis (Pan), Pantoea agglomerans (Pag), Pantoea vagans (Pva) and Rhodobacter sphaeroides (Rsp). The sequence for those genes are shown in SEQ ID NO: 2 (PanE crtE), SEQ ID NO: 3 (PagE crtE), SEQ ID NO: 4 (PvaE crtE), SEQ ID NO: 5 (RspE crtE), SEQ ID NO: 6 (PanB crtB), SEQ ID NO: 7 (PagB crtB), SEQ ID NO: 8 (PvaB crtB), SEQ ID NO: 9 (RspB crtB), SEQ ID NO: 10 (PanI crtI), SEQ ID NO: 11 (PagI crtI), SEQ ID NO: 12 (PvaI crtI), and SEQ ID NO: 13 (RspI crtI).

The coding sequence of idi (SEQ ID NO: 14) was from the genome of the E. coli strain MG1655 and served as a reporter gene for identifying positive clones. The BsaI recognition sites in all the above sequence were removed by introducing silent mutations. The resulting donor sequences were then cloned into pUC57 donor vectors.

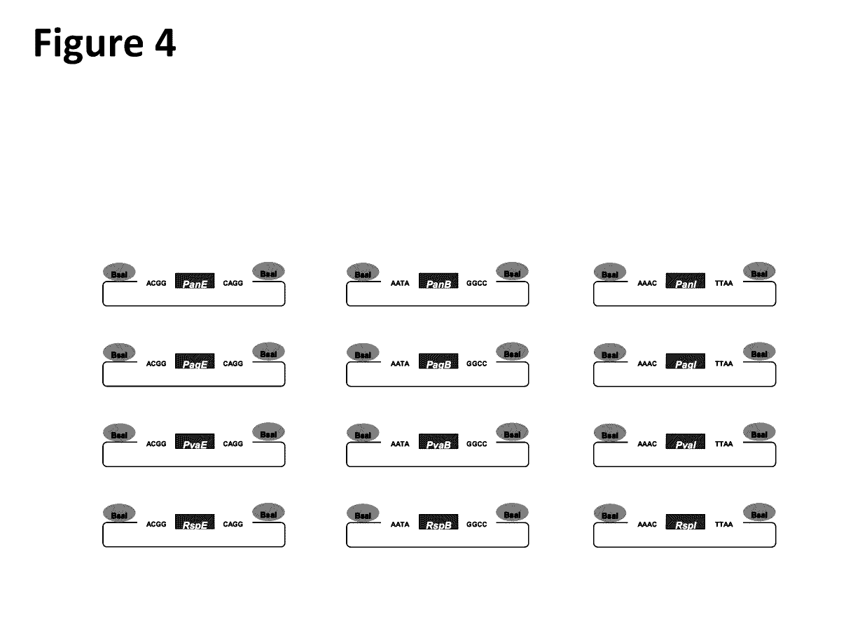

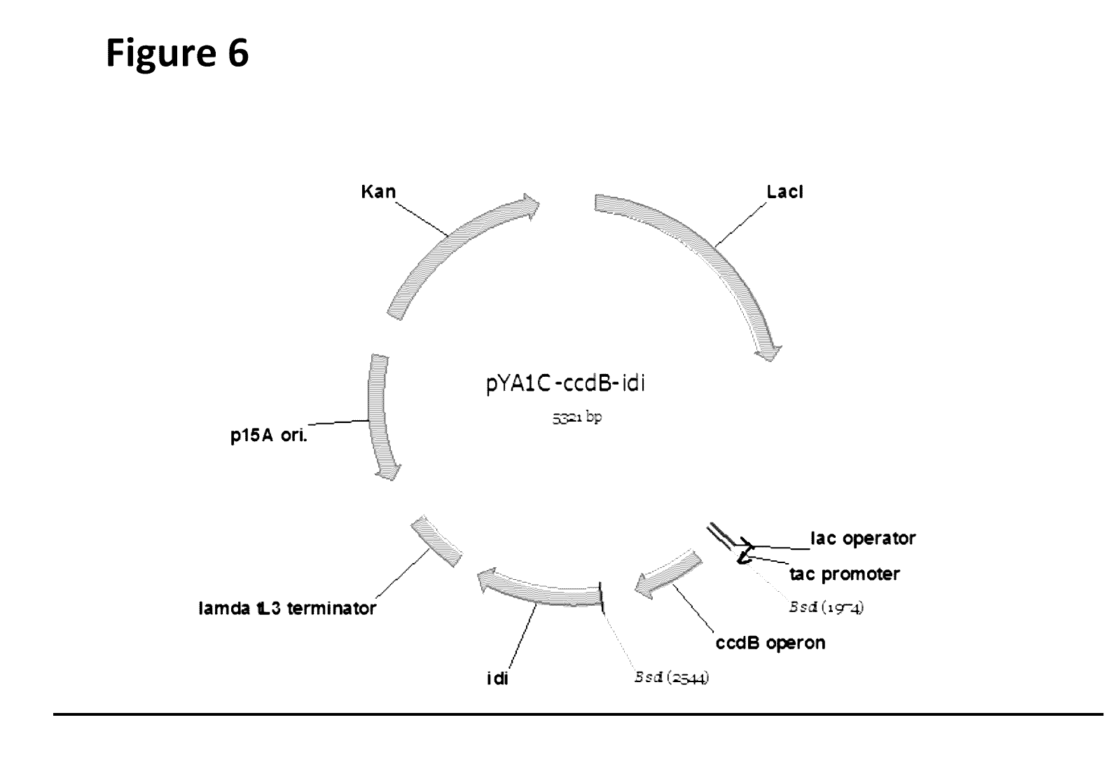

As can be seen in FIG. 4, the 5′ overhangs for crtE, crtB, crtI, and idi were ACGG, AATA, AAAC, and CAAA, respectively. Only one version of the idi gene was used in the assembly, and its coding sequence was cloned into the pYC1k-ccdB vector to generate a pYC1k-ccdB-idi vector, shown in FIG. 6, with a full length sequence shown in SEQ ID NO: 15.

Twenty different RBS sequences were designed for each gene. A schematic of how double-stranded oligo-linker nucleic acid molecules, containing RBS encoding sequences, were used to assemble the 4 different genes in 6 different gene orders is shown in FIG. 5.

The OLMA assembly product was transformed into Trans-T1 cells for expression analysis. Different clones displayed different intensities of red coloring, and this readout was used to determine the level of lycopene production of the clones. The lycopene production of 90 randomly isolated colonies ranged from 1.15 to 11.24 mg/g. These results indicated that (a) genes from different species, (b) different RBS strengths, and (c) different gene orders could all, to some extent, affect gene expression and therefore metabolic pathway efficiency. The OLMA method made it possible to balance the expression level of the metabolic pathway genes by combinatorially adjusting all three factors simultaneously.

As demonstrated by Example 2, the OLMA method allows one-step assembly of variants of multiple genes and variants of multiple RBS sequences in various orders and thus enables simultaneously tuning the expression of several genes. Double-stranded oligo-linker nucleic acid fragments containing RBS encoding sequences were used not only as linkers for the assembly, but also as regulatory sequences to control gene expression levels. Features of the OLMA method, such as using linker overhangs to determine assembly order and one-step assembly to construct combinatorial plasmid libraries, allow high throughput metabolic or biological pathway optimization, and improves subsequent strain engineering.

The invention has been used to optimize the lycopene production pathway and can readily be expanded to optimize other metabolic or biological pathways.

While the invention has been described in detail, and with reference to specific embodiments thereof, it will be apparent to one of ordinary skill in the art that various changes and modifications can be made therein without departing from the spirit and scope of the invention.

1. A method for generating a library of expression vectors comprising a plurality of donor sequences, the method comprising:
(a) obtaining a plurality of donor vectors, each independently comprising: (i) a first cleavage site recognizable by a type IIS restriction endonuclease, (ii) a donor sequence, and (iii) a second cleavage site recognizable by the type IIS restriction endonuclease, wherein upon digestion with the type IIS restriction endonuclease, the plurality of donor vectors will provide a plurality of double-stranded donor nucleic acid fragments, each independently comprising: (i) a donor 5′ overhang, (ii) a donor sequence, and (iii) a donor 3′ overhang, and the donor 5′ overhang and the donor 3′ overhang are not complementary to each other;
(b) providing an entry vector comprising a selectable marker gene and a first cleavage site and a second cleavage site recognizable by the type IIS restriction endonuclease, wherein upon digestion with the type IIS restriction endonuclease, the entry vector will provide an entry vector backbone comprising: (i) an entry vector 5′ overhang, (ii) an entry vector backbone comprising the selectable marker gene, and (iii) an entry vector 3′ overhang;
(c) providing a plurality of chemically synthesized double-stranded oligo-linker nucleic acid molecules, each independently comprising: (i) a linker 5′ overhang, (ii) a linker sequence, and (iii) a linker 3′ overhang, wherein the linker 5′ overhang is complementary to at least one of the donor 3′ overhangs or to the entry vector 3′ overhang, and the linker 3′ overhang is complementary to at least one of the donor 5′ overhangs or to the entry vector 5′ overhang;
(d) mixing (i) the plurality of donor vectors, (ii) the plurality of double-stranded oligo-linker nucleic acid molecules, (iii) the entry vector, (iv) the type IIS restriction endonuclease, and (v) a ligase, in a reaction mixture; and
(e) incubating the reaction mixture under a condition to assemble the library of expression vectors.

2. The method of claim 1, wherein the plurality of donor vectors and the entry vector do not contain additional cleavage sites recognizable by the type IIS restriction endonuclease.

3. The method of claim 1, wherein each of the donor 5′ overhang, the linker 5′ overhang, the entry vector 5′ overhang, the donor 3′ overhang, the linker 3′ overhang and the entry vector 3′ overhang has 4 nucleotides.

4. The method of claim 1, wherein each of the donor DNA sequences comprises at least 200 base pairs.

5. The method of claim 1, wherein each of the double-stranded oligo-linker nucleic acid molecules comprises no more than 50 base pairs.

6. The method of claim 1, wherein each of the double-stranded oligo-linker nucleic acid molecules comprises a pair of phosphorylated chemically synthesized oligonucleotides.

7. The method of claim 1, wherein the donor sequences comprise coding sequences of polypeptides and the linker sequences comprise regulatory sequences.

8. The method of claim 1, wherein the condition in step (e) comprises:
i) 10 cycles of 5 minutes at 37° C. followed by 10 minutes at 16° C.;
ii) 15 minutes at 37° C.;
iii) 5 minutes at 50° C.; and
iv) 5 minutes at 80° C.

9. The method of claim 1, further comprising:
a) treating the library of expression vectors with DNase; and
b) transforming the DNase-treated library of expression vectors into competent cells.

10. A system for generating a library of expression vectors comprising a plurality of donor sequences, the system comprising:
(a) a plurality of donor vectors, each independently comprising: (i) a first cleavage site recognizable by a type IIS restriction endonuclease, (ii) a donor sequence, and (iii) a second cleavage site recognizable by the type IIS restriction endonuclease, wherein upon digestion with the type IIS restriction endonuclease, the plurality of donor vectors will provide a plurality of double-stranded donor nucleic acid fragments, each independently comprising: (i) a donor 5′ overhang, (ii) a donor sequence, and (iii) a donor 3′ overhang, and the donor 5′ overhang and the donor 3′ overhang are not complementary to each other;
(b) an entry vector comprising a selectable marker gene and a first cleavage site and a second cleavage site recognizable by the type IIS restriction endonuclease, wherein upon digestion with the type IIS restriction endonuclease, the entry vector will provide an entry vector backbone comprising: (i) an entry vector 5′ overhang, (ii) an entry vector backbone comprising the selectable marker gene, and (iii) an entry vector 3′ overhang;
(c) a plurality of chemically synthesized double-stranded oligo-linker nucleic acid molecules, each independently comprising: (i) a linker 5′ overhang, (ii) a linker sequence, and (iii) a linker 3′ overhang, wherein the linker 5′ overhang is complementary to at least one of the donor 3′ overhangs or to the entry vector 3′ overhang, and the linker 3′ overhang is complementary to at least one of the donor 5′ overhangs or to the entry vector 5′ overhang; and
(d) the type IIS restriction endonuclease and a ligase to be mixed and incubated with the plurality of donor vectors, the plurality of double-stranded oligo-linker nucleic acid molecules, and the entry vector for the assembly of the library of expression vectors.

11. The system of claim 10, wherein the plurality of donor vectors and the entry vector do not contain additional cleavage sites recognizable by the type IIS restriction endonuclease.

12. The system of claim 10, wherein each of the donor 5′ overhang, the linker 5′ overhang, the entry vector 5′ overhang, the donor 3′ overhang, the linker 3′ overhang and the entry vector 3′ overhang has 4 nucleotides.

13. The system of claim 10, wherein each of the donor DNA sequences comprises at least 200 base pairs.

14. The system of claim 10, wherein each of the double-stranded oligo-linker nucleic acid molecules comprises no more than 50 base pairs.

15. The system of claim 10, wherein each of the double-stranded oligo-linker nucleic acid molecules comprises a pair of phosphorylated chemically synthesized oligonucleotides.

16. The system of claim 10, wherein the donor sequences comprise coding sequence for polypeptides and the linker sequences comprise or encode regulatory sequences.

17. The system of claim 10, further comprising DNase.

18. A method for optimizing a biological pathway, comprising:
(a) generating a library of expression vectors using a method of claim 1, wherein the library comprises a plurality of genes of the biological pathway or variants thereof as the donor sequences, and a plurality of regulatory sequences as the linker sequences;
(b) transforming the library of expression vectors into a host cell; and
(c) identifying clones having the optimized biological pathway from the transformed cells.

19. The method of claim 18, wherein the biological pathway is a lycopene biosynthetic pathway, the library of expression vectors contains the donor sequences comprising crtE, crtB, crtI, and idi genes, and the linker sequences encoding ribosomal binding sites (RBSs).

20. The method of claim 19, wherein the donor sequences comprises the criE, crtB, crtI, and idi genes from different species, the linker sequences encode RBSs with different strength, and the library of expression vectors contains the genes and the RBSs in different orders.
